(19) United States
(12) Patent Application Publication
(10) Pub. No.: US 2014/0228528 A1
(43) Pub. Date: Aug. 14, 2014

(54) POLYGUANIDINE SILICATE AND USE THEREOF

(71) Applicants: Oskar Schmidt, Wien (AT); Nikita Schmidt, Wien (AT); Christoph Schmidt, Graz (AT)

(72) Inventors: Oskar Schmidt, Wien (AT); Nikita Schmidt, Wien (AT); Christoph Schmidt, Graz (AT)

(73) Assignee: MINDINVEST HOLDINGS LTD., Valletta VLT (MT)

(21) Appl. No.: 13/639,967

(22) PCT Filed: Nov. 2, 2011
(86) PCT No.: PCT/EP2011/005515

§ 371 Date: Mar. 5, 2014

(51) Int. Cl. C08G 73/02; A01N 47/44; A61K 31/785

(52) U.S. Cl. CPC C08G 73/0213; C08G 73/024; C08G 73/0206; A61K 31/785; A01N 47/44
USPC 525/540

(57) ABSTRACT

A polyguanidine silicate obtainable by reacting a polymeric guanidine salt provided in an aqueous solution with an aqueous solution of a sodium and/or potassium silicate.

The present invention relates to a polyguanidine silicate as well as to the manufacture and use thereof. Furthermore, the present invention relates to drug compositions which contain a polyguanidine silicate as a drug substance.

Biocidal polymeric guanidine salts based on diamines are known, inter alia, from AT 406.163 B and AT 411.060 B. Chlorides of these polymers are produced by reacting the diamine, e.g., hexamethylene diamine or triethylene glycol diamine, with guanidine hydrochloride. A cationic polymer (polyguanidinium cation) thereby forms with chloride as the counterion. It is known that said compound has pronounced biocidal properties.

Further salts of these polymeric guanidines can be produced according to AT 411.060 B in that, instead of the hydrochloride, a different salt of guanidine is used. In AT 411.060 B, the cationic polymers (polyguanidinium cations) are produced in this way with dihydrogen phosphate, carbonate, nitrate, dehydroacetate or citrate as the counterion. Example 9 of AT 411.060 B indeed relates to the manufacture of a silicate by reacting triethylene glycol diamine with guanidine silicate, whereby polytriethylene glycol guanidine silicate is supposed to form. However, it has been shown that said reaction does not work as described in AT 411.060 B and a polymeric guanidine silicate cannot be produced. But a silicate would be desirable since it burdens the environment less during its application than other polymeric guanidines.

From RU 2 236 428 C1, a coating material is known which is used for disinfection and contains the following ingredients: chlorosulfonated polyethylene, polyhexamethylene guanidine, water, an oganic solvent and dialkyl phosphoric acid. Furthermore, said composition may contain 0.1-0.3% sodium silicate.

From JP 2009108184 A, a bactericidal detergent composition is known which comprises: 0.1-5% by mass of a polyhexamethylene guanidine salt, 0.5-3% by mass of a silicate, 1-10% by mass of a first alkylene oxide adduct from a secondary alcohol, and 1-10% of a second alkylene oxide adduct.

GB 1 202 303 describes a guanidine silicate having a molar ratio of guanidinium ions/silicate ions of 1.5-0.65. A polymeric product is also described which can be obtained by polymerizing said guanidine silicate, e.g., with formaldehyde.

From WO 2009/009815, a silicate filler for synthetic materials is known which is modified with a polymeric guanidine derivative acting as a biocide. Said filler is produced by mixing, e.g., a 1-30% aqueous solution of the polymeric guanidine hydrochloride at room temperature into fine aerosil types provided as solids. During said mixing, the polymeric guanidine hydrochloride binds to the silicate present in solid form. Subsequently, the water is removed by drying. The binding of the polymeric guanidine derivatives to the silicate is so firm that they are virtually no longer water-soluble, but still display their microbicidal activity. Without being bound to any particular theory, it is stated that the hydrochloride, after it has bound to the silicate, is still provided as such, i.e., the counterion to the cationic guanidine is still the chloride. In other words, this is not a polyguanidinium silicate, that is, a cationic polyguanidinium with a silicate as the counterion.

Polymers based on guanidinium hydrochloride and acting as microbiocides, in particular their activity against Escherichia coli bacteria, are likewise already known (cf. WO 01/85676). Furthermore, it is already known that such guanidine derivatives can be used as fungicidal agents (cf. WO 2006/047800). The polymers Akacid®, the poly-[2-(2-ethoxy)-ethoxyethyl-guanidinium chloride], and Akacid plus®, a 3:1-mixture of poly-(hexamethylene guanidinium chloride) and poly-[2-(2-ethoxy)-ethoxyethyl)-guanidinium chloride], are of particular significance (cf. Antibiotika Monitor, 22nd Volume, Issue Jan. 2, 2006, Online Edition under http://www.antibiotikamonitor.at/06_{—}12/06_{—}12_inhalt.htm).

The aforesaid polymers which act as microbiocides belong to the group of cationic antiseptics which comprise substances that are very diverse in chemical terms, but have, as a common characteristic, strongly basic groups bound to a rather bulky lipophilic molecule. The most important representatives among the quarternary ammonium compounds are benzalkonium chloride and cetrimide, among the bisbiguanides chlorhexidine and alexidine and among the polymeric biguanides polyhexamethylene biguanide (PHMB).

Because of their own positively charged molecules, substances with cationically antimicrobial activity display a high binding affinity toward the negatively charged cell walls and membranes of bacteria. The result of disturbing those access points will first be a decrease in membrane fluidity and a failure of osmoregulatory and physiological cell functions. In further consequence, hydrophilic pores emerge in the phospholipide membrane, and the protein function is disrupted. The final result is a lysis of the target cell. This membrane-impairing mode of action could also be demonstrated for polymeric guanidines against Escherichia coli.

From WO 99/54291, polyhexamethylene guanidines are known which, as a result of their microbicidal activity, can be used as disinfectants. These substances are produced by polycondensation of guanidine with an alkylene diamine, in particular hexamethylene diamine. The condensation product obtained has a good biocidal activity.

From WO 2006/047800 A1, a polymeric condensation product is known which can be obtained by reacting guanidine or the salt thereof with an akylene diamine and an oxyalkylene diamine. Said condensation product acts as a biocide and in particular as a fungicide. A representative of this condensation product is marketed also as “Akacid Plus”.

On the other hand, from WO 2008/080184 A2, the manufacture and use of polymeric guanidinium hydroxides is known for control of microorganisms, which guanidinium hydroxides are based on a diamine containing oxyalkylene chains and/or alkylene groups between two amino groups and obtainable by polycondensing a guanidine acid addition salt with the diamine, whereby a polycondensation product in the form of a salt is obtained, which is subsequently converted into the hydroxide form by means of basic anion exchange.

In the prior art, drug compositions are furthermore described which contain the polymeric guanidine derivatives as drug substances, have antimicrobial activity and can be used in human medicine as well as in veterinary medicine for fighting infections.

When the drug compositions were used, e.g., in the veterinary field, it became apparent that poultry rejected drinking water to which the polymeric guanidine derivative had been added. Something similar was observed during the feeding of pigs, namely when granular polymeric guanidine derivative was admixed to the pig fodder.

A further disadvantage which became apparent in resorption studies that had been carried out is that up to 17% of the active substance has been resorbed from the gastro-intestinal tract.

The object of the present invention is to provide a polymeric guanidine salt which has biocidal activity, is easy to produce and does not have the above-mentioned disadvantages. Furthermore, the polyguanidine salt according to the invention should be as sparingly water and alcohol soluble as possible.

Said object is achieved with a polyguanidine silicate which is obtainable by mixing a first aqueous solution containing a salt of a polymeric guanidine with an inorganic or organic acid in a dissolved state with a second aqueous solution containing sodium and/or potassium silicate in a dissolved state, whereby the polyguanidine silicate forms as a solid as well as a sodium and/or potassium salt of the inorganic or organic acid, which salt is provided in dissolved form.

The polyguanidine silicate precipitating as a solid can simply be filtered out of the reaction mixture. By washing, it can be freed from starting products, which possibly are still present, and from sodium and/or potassium salt of the inorganic or organic acid, which possibly is still present. A chemical analysis of the product purified in this way showed that virtually no chloride was still present, that all chloride ions had thus been replaced by silicate ions.

As already described above, water-soluble polymeric guandine salts which are used for the synthesis of the polyguanidine silicate according to the invention are known in the art. Preferred representatives of this class of compounds will be described further below.

It is crucial that the polymeric guandine salt is provided in an aqueous solution and is reacted with an aqueous solution of a sodium and/or potassium silicate. This can be done by simple mixing, whereby the polyguanidine silicate according to the invention immediately precipitates as a powder from the aqueous solution.

In the present invention, “water glass” is preferably used as an aqueous solution of a sodium and/or potassium salt. Commercially available aqueous solutions of alkali silicates are generally referred to as “water glass”, are obtained by dissolving the melt obtained from silica sand and potash or from silica sand and soda or, respectively, Glauber's salt/carbon (“soda water glass”) in water and mainly contain the salts M₂SiO₃ and M₂Si₂O₅ (Holleman-Wiberg, “Lehrbuch der anorganischen Chemie”, 1964, p. 330).

A polymeric bisguanidine salt will preferably be used as the polymeric guanidine salt. A preferred representative of bisguanidine salts is polyhexamethylene biguanide (polyhexanide) as known in the prior art.

A further preferred embodiment of the polyguanidine silicate according to the invention consists in that the polymeric guanidine salt is obtainable by reacting a guanidine salt with an alkylene diamine and/or an oxyalkylene diamine Such polymeric guanidine salts are known, for example, from AT 406.163 B, AT 408.302 B, AT 411.060 B and WO 2006/047800 A1.

A preferably used polymeric guanidine salt is obtainable via a reaction in which, per mole of diamine (sum of alkylene diamine and oxyalkylene diamine), 0.8 to 1.2 moles of guanidine salt thereof are used.

A further preferably used polymeric guanidine salt is obtainable via a reaction in which the alkylene diamine and the oxyalkylene diamine are used at a molar ratio of between 4:1 and 1:4.

The amino groups of the alkylene diamine and/or the oxyalkylene diamine are preferably terminal.

Furthermore, a compound of general formula

NH₂(CH₂)ₙNH₂

is preferably provided as the alkylene diamine, wherein n is an integer between 2 and 10, in particular 6.

Furthermore, a compound of general formula

NH₂[(CH₂)₂O)]ₙ(CH₂)₂NH₂

is preferably provided as the oxyalkylene diamine, wherein n is an integer between 2 and 5, in particular 2.

In particular, triethylene glycol diamine (relative molecular mass: 148), polyoxypropylene diamine (relative molecular mass: 230) and/or polyoxyethylene diamine (relative molecular mass: 600) is/are used as the oxyalkylene diamine.

The average molecular mass of the polymeric guanidine salt used ranges between 500 and 3,000.

A hydrochloride is preferably provided as the salt of the guanidine.

The polyguanidine silicate according to the invention has a pronounced biocidal activity and can be used as a biocidal agent or as an additive with biocidal activity.

The polyguanidine silicate according to the invention can be added, for example, to paints, lacquers, silicone substances, other building materials, synthetic materials or cosmetics in order to protect them from harmful microbes and/or to prevent the spreading of such undesirable germs.

The present invention achieves the protection which is sought by incorporating the biocide according to the invention in particular in powder form. The biocide according to the invention provides a substantial advantage in that it is not water-soluble. In this way, materials with antimicrobial activity are produced with as little environmental impact as possible.

Furthermore, the biocide according to the invention cannot reach the groundwater. The silicate as the major component of the earth's surface is not harmful.

Also, in production processes which utilize a lot of water such as, e.g., in the paper industry, the biocide according to the invention can be added to the other fillers during the manufacturing process, for example, in powder form and can thus protect, e.g., cardboard articles from mould infestation and degradation.

Admixed to dispersion paints, silicone joint sealers and other coating materials, the biocide according to the invention achieves its object of equipping the materials in an antimicrobial fashion.

The addition of biocides is basically known, but so far they have exhibited the disadvantage of water solubility. However, the polyguanidine silicate according to the invention is not water-soluble. This is a very crucial advantage.

As already described above, the manufacture occurs in an aqueous phase, wherein either the polyguanidine salt or the solution of the silicate, in particular water glass, is presented and the reactant is slowly added under vigorous stirring. Upon addition of the reactant, the polyguanidine silicate according to the invention immediately precipitates, with a potassium or sodium salt forming, which remains in the aqueous solution.

A polymeric bisguanidine salt will preferably be used as the polymeric guanidine salt. A preferred representative of bisguanidine salts is polyhexamethylene biguanide (polyhexanide) as known in the prior art.

A further preferred embodiment of the polyguanidine silicate contained in the drug composition according to the invention consists in that the polymeric guanidine salt is obtainable by reacting a guanidine salt with an alkylene diamine and/or an oxyalkylene diamine. Such polymeric guanidine salts are known, for example, from AT 406.163 B, AT 408.302 B, AT 411.060 B and WO 2006/047800 A1.

A preferably used polymeric guanidine salt is obtainable via a reaction in which, per mole of diamine (sum of alkylene diamine and oxyalkylene diamine), 0.8 to 1.2 moles of guanidine salt thereof are used.

A further preferably used polymeric guanidine salt is obtainable via a reaction in which the alkylene diamine and the oxyalkylene diamine are used at a molar ratio of between 4:1 and 1:4.

The amino groups of the alkylene diamine and/or the oxyalkylene diamine are preferably terminal.

Furthermore, a compound of general formula

NH₂(CH₂)ₙNH₂

is preferably provided as the alkylene diamine, wherein n is an integer between 2 and 10, in particular 6.

Furthermore, a compound of general formula

NH₂[(CH₂)₂O)]ₙ(CH₂)₂NH₂

is preferably provided as the oxyalkylene diamine, wherein n is an integer between 2 and 5, in particular 2.

In particular, triethylene glycol diamine (relative molecular mass: 148), polyoxypropylene diamine (relative molecular mass: 230) and/or polyoxyethylene diamine (relative molecular mass: 600) is/are used as the oxyalkylene diamine.

The average molecular mass of the polymeric guanidine salt used ranges between 500 and 3,000.

A hydrochloride is preferably provided as the salt of the guanidine.

The polyguanidine silicate contained as a drug substance in the drug composition according to the invention has a pronounced biocidal activity and can be used as a biocidal agent or as an additive with biocidal activity.

Furthermore, the biocide according to the invention cannot reach the groundwater. The silicate as the major component of the earth's surface is not harmful.

As already described above, the manufacture occurs in an aqueous phase, wherein either the polyguanidine salt or the solution of the silicate, in particular water glass, is presented and the reactant is slowly added under vigorous stirring. Upon addition of the reactant, the polyguanidine silicate according to the invention immediately precipitates, with a potassium or sodium salt forming, which remains in the aqueous solution.

It has turned out that the polyguanidine silicate according to the invention is virtually not water-soluble, liposoluble and also not alcohol soluble. It is all the more surprising that the polyguanidine silicate according to the invention still displays its biocidal activity (also see below). Moreover, it is tolerated well by humans and animals upon oral ingestion.

Because of all these properties, the polyguanidine silicate according to the invention is suitable also as an additive in foodstuffs in order to be able to preserve them better.

A further field of application is animal feed to which the polyguanidine silicate according to the invention can be added. Besides, in this way, the antibiotics which tend to be used in factory farming even though their use increasingly gets banned in more and more countries can be replaced. The polyguanidine silicate according to the invention can also be used in fish breeding (“fish farming”).

Since the polyguanidine silicate according to the invention displays its biocidal activity also in humans and animals, a further preferred embodiment of the present invention is a drug composition which contains the polyguanidine silicate according to the invention as a drug substance. The drug composition according to the invention is particularly suitable for fighting infections, namely in humans and animals.

With the following examples, preferred embodiments of the invention are described in even greater detail, wherein, in Example 1, the manufacture of a preferred representative of the polyguanidine silicate according to the invention is described. Examples 2 to 6 demonstrate the properties of the polyguanidine silicate manufactured in Example 1.

### EXAMPLE 1

For the manufacture of a polyguanidine silicate according to the invention, the polymeric guanidine salt known from AT 406.163 B was used, namely polyhexamethylene guanidine hydrochloride.

In a 50 L barrel, 24 l of an aqueous 1% solution of polyhexamethylene guanidine hydrochloride were presented. The manufacture of the polyhexamethylene guanidine hydrochloride was effected according to the method described in AT 406.163 B.

1.5 l of a 20% solution of sodium water glass was slowly (over approx. 2 h) dropped into this solution by means of a dropping funnel while being stirred. In this process, the substance according to the invention precipitated as a white powder. Said powder can be separated in various ways. In doing so, the powder may also be washed with water, if necessary, in order to remove the sodium chloride which has formed, together with washing out starting substances which possibly are still present.

The powder was filtered off and the filter cake was dried in the drying cabinet. By chemical analysis it was demonstrated that the product exhibited virtually no more detectable chloride, that all chloride ions of the polyguanidine chloride had thus been replaced by silicate ions.

It has been shown that the method according to the invention is properly and economically feasible also on an industrial scale.

The powder obtained according to Example 1 was examined with regard to oral toxicity. The method was employed according to OECD Guideline 423, 1996, and Directive 96/54/EC, method Bitris. The powder was suspended in deionized water and administered once to six male and six female rats (Crl:CD(SD)IGS BR) via stomach intubation. The result: LD 50 oral of PGS as an active substance is higher than 5000 mg/kg body weight. No toxic effects were observed.

The antimicrobial and biocidal activities, respectively, of the powder according to the invention were tested and described in the following examples.

### EXAMPLE 2

In this example, the bactericidal activity of the powder described in Example 1 (in the following, referred to as “PGS”) in Muller Hinton Bouillon (MHB) against the bacterium Escherichia coli ATCC 10536 is documented.

### Material and Method

For testing the bactericidal effectiveness of the PGS, an experiment was performed in test tubes with screw caps in order to determine the minimum inhibition concentration (MHK). The respective dilution series were tested with Muller Hinton Bouillon mixed with E. coli at 10⁵ KBE/mL. In each case, 10 ml of the liquids were pipetted into test tubes (20 ml).

Since PGS is not water-soluble and the powder settled in a short time at the bottom of the test tubes, the test tubes were incubated at 35° C. over night in the dark while lying on a shaker. In this way, the PGS particles were kept moving and thus came into sufficient contact with the bacteria.

After the first evaluation, the samples were incubated for further 96 hours at room temperature (20° C.±2° C.). Clouding of the transparent starting liquids indicates bacterial growth. The lowest concentration at which no bacterial growth occurs, i.e., the liquid remains transparent, indicates the minimum inhibition concentration.

The dilution series were produced in 3 replicates at concentrations of 0, 1, 5, 10, 50, 100 ng/mL. PGS 11 and Muller Hinton Bouillon without additive as a control were tested for bacterial growth. In Table 1, the results of the experiment are summarized.

TABLE 1
determination of the minimum inhibition concentration of PGS against
Escherichia coli (x = bacteria grow; ∘ = bacteria do not grow).

| | concentration (μg of powder per mL of MHB) | | | | |
|---|---|---|---|---|---|
| MHB additive | 1 | 5 | 10 | 50 | 100 |
| Control (MHB without | x | x | x | x | x |
| additive) | | | | | |
| PGS | x | x | ∘ | ∘ | ∘ |

1. Müller Hinton Bouillon: The control for bacterial growth was positive, i.e., the liquids were cloudy in all 3 test tubes.
2. PGS: At a concentration of 1 μg/mL PGS and 5 μg/mL, the liquids in the test tubes were cloudy, i.e., bacteria did grow there. But at concentrations of 10, 50 and 100 μg/mL, bacterial growth did no longer occur. Thus, the minimum inhibition concentration in this dilution series was 10 μg/mL.

In Table 1, it can be seen that the PGS has a good bactericidal activity, with the minimum inhibition concentration ranging between 5 and 10 μg/ml.

### EXAMPLE 3

In this example, the fungicidal activity of PGS incorporated in potato dextrose agar against the mould fungi Aspergillus brasiliensis (niger) DSM 1957 and Penicillium funiculosum (pinophilum) DSM 1944 is described.

Mould fungi occur in great diversity anywhere in the environment, among which the genera Aspergillus and Penicillium occur most frequently as mould creators in interior spaces. The fungi Aspergillus brasiliensis (niger) DSM 1957 and Penicillium pinophilum (funiculosum) DSM 1944 were selected for testing the fungicidal activity of PGS against those microorganisms.

Material and Method

The fungi Aspergillus brasiliensis DSM 1957 and Penicillium pinophilum DSM 1944 were cultivated on a potato dextrose agar substrate in Petri dishes (diameter 90 mm) at 24° C. in the dark. After two weeks, aqueous spore solutions were produced from the well-growing and sporulating fungal cultures and were adjusted to a spore concentration of, in each case, 10⁴ spores/mL by means of a haemocytometer.

The two spore solutions of Aspergillus brasiliensis and Penicillium pinophilum were distributed in an unmixed state or a state of being mixed 1:1 on the surfaces of a fresh potato dextrose agar substrate in Petri dishes by means of a hand sprayer (30ml) in such a way that fine droplets formed on the surfaces without running together. The tested concentrations of the PGS in agar were determined to be 0, 10, 20, 40 and 80 μg/ml. All treatments were tested in three replicates. The fungal growth was assessed at weekly intervals.

The results are indicated in Table 2. In Table 2, it can be seen that the PGS displayed a fungicidal activity against the tested fungi in the potato dextrose agar substrate after 21 days at all tested concentrations. No mycelium growth could be observed on the agar surface. In the control without PGS, the Petri dishes were completely overgrown by fungus mycelium already after one week. This means that PGS at less than 10 μg/mL is able to stop these fungi from growing.

TABLE 2
Antimicrobial effectiveness of PGS incorporated in a potato dextrose
agar substrate against the mould fungi Aspergillus brasiliensis and
P. pinophilum, in an unmixed state or a state of being mixed 1:1,
21 days after inoculation, (x = fungi grow; ∘ = fungi do not grow).

| Concentration | | | A. brasiliensis + |
|---|---|---|---|
| (μg PGS per mL) | A. brasiliensis | P. pinophilum | P. pinophilum |
| 0 | x | x | x |
| 10 | ∘ | ∘ | ∘ |
| 20 | ∘ | ∘ | ∘ |
| 40 | ∘ | ∘ | ∘ |
| 80 | ∘ | ∘ | ∘ |

### EXAMPLE 4

In this example, the fungicidal activity of PGS in an acrylic interior dispersion paint against the mould fungi Aspergillus brasiliensis (niger) DSM 1957 and Penicillium pinophilum (funiculosum) DSM 1944 is described.

### Material and Method

Sax Walith Power acrylic interior dispersion paint is a commercially available water-dilutable acrylic dispersion lacquer of the firm Sax Farben AG, CH Urdorf. The powder according to the invention was stirred homogeneously into the dispersion paint at a final concentration of 1% (w/w). Subsequently, the viscous paint and the colour mixture were coated with a brush onto, in each case, four filter paper sheets (5cm×5cm) in a uniform layer. For drying, these coatings were stored at 22° C. for 24 hours.

The fungicidal effectiveness of the dry paint surfaces was accomplished with the test germs Aspergillus brasiliensis DSM 1957 and Penicillium pinophilum DSM 1944 following the standard method of the “American Society for Testing and Materials” ASTM D 5590 (2005) “Determining the resistance of paint films and related coatings to fungal defacement by accelerated four-week agar plate assay”. The more the fungi grow, the less is the effectiveness of the material to be tested. For evaluation, the paint samples were placed on a potato dextrose culture medium located in Petri dishes (diameter 90 mm) Medium and samples were then inoculated with a spore solution of the two test germs.

For this purpose, the fungi which had been divided up according to species were cultivated on a malt extract agar substrate in Petri dishes (diameter 90mm) at 24° C. in the dark. After two weeks, aqueous spore solutions were produced from the well-growing and sporulating fungal cultures and were adjusted to a spore concentration of, in each case, 10⁴ spores/mL by means of a haemocytometer. The two spore solutions were mixed 1:1 and distributed on the sample surfaces and the uncovered areas of the culture medium by means of a hand sprayer (30 mL) in such a way that fine droplets formed on the surfaces.

The visual evaluation of the fungal growth was conducted for one month at weekly intervals according to the following scale:

0=no fungal growth on the plates

1=<10% of the plate covered with fungi (traces)

2=10-30% of the plate covered with fungi (little growth)

3=>30-60% of the plate covered with fungi (medium growth)

4=>60-100% of the plate covered with fungi (strong growth)

If values equal to or smaller than 1 occur, the test substance is regarded as fungistatic.

The results of the fungicidal effectiveness of the paint surfaces are illustrated in Table 3. The fungal growth on the sample surfaces without (0%) and with PGS (1%) became visible in the first week after inoculation. In the subsequent weeks, the test fungi on the samples without PGS developed substantially more than those on the samples with PGS, which became clearly evident also in the numerical values of Table 3. The fungal growth inhibiting activity of the PGS persisted until the end of the experiment, four weeks after inoculation.

The samples enriched with PGS were colonized by the test fungi only from the margins. The potato dextrose culture medium not covered with test lamellae in the Petri dishes was completely overgrown by fungus mycelium from the first week.

TABLE 3
Determination of the fungicidal activity of Sax Walith Power interior
dispersion paint with or without PGS powder additive on filter
paper sheets (5 × 5 cm) against Aspergillus brasilensis and Penicillium
pinophilum following ASTM international: D 5590 (2005),
n = 4. Total duration of the experiment = 4 weeks.

| | rating value | | | |
|---|---|---|---|---|
| Paint composition | week 1 | week 2 | week 3 | week 4 |
| Sax Walith Power without PGS | 3.0 | 3.8 | 3.8 | 3.8 |
| Sax Walith Power + 1% PGS | 0.8 | 1.0 | 1.3 | 1.0 |

### EXAMPLE 5

With this infection experiment, the microbicidal activity of the polyguanidine silicate according to the invention was tested in chicken with regard to a representative of Enterobacteriacaea, notably Campylobacter jejuni.

### Material and Methods

Animals and Infection

Pathogen-free (SPF) chicks of the breed VALO (Lohmann, Cuxhaven) were incubated at the Klinik für Geflügel, Ziervogel, Reptilien and Fische, Veterinärmedizinische Universität Wien, and kept in insulators under SPF conditions. For the present study, 60 animals were kept separately in four groups (15 animals each). At the beginning of the experiment, the animals were marked individually by Swiftack.

The infection of the animals was effected orally with 1×10⁸ KBE/animal on the 14th day of their lives. The bacterial isolate used was a strain provided as a pure culture at the Klinik für

Geflügel, Ziervögel and Reptilien, which had also already been used in earlier experiments. The PGS was administered to the animals twice a day at a total concentration of 500mg/kg body weight by means of a crop probe.

The killing was performed in accordance with animal protection laws by euthanasia or by neck blows, with bleeding.

Group compositions and samplings The following group composition of the chicks was effected in order to examine the effect of PGS on the infective agent Campylobacter jejuni as well as the health status of the animals:

Group 1: medication with PGS and infection with Campylobacter jejuni

Group 2: medication with PGS and no infection with Campylobacter jejuni

Group 2: without medication and infection with Campylobacter jejuni

Group 4: without medication and no infection with Campylobacter jejuni

### Bacteriological Examination of Cloacal Swabs

Cloacal swabs for verifying freedom from bacteria were taken from all animals on the 14th day of their lives. The taking of cloacal swabs on the 21st and 28th days of their lives was conducted, in each case, on 5 animals per group and served, on the one hand, for determining the bacterial secrection rate of the animals infected with C. jejuni as well as for demonstrating freedom from bacteria in the non-infected animals. The examinations were performed via the bacterial enrichment method.

### Results

### General Behaviour and Health Status of the Chicks

No significant difference in general behaviour/health status could be detected between animals which had been given PGS and animals which had received no preparation (negative control group).

TABLE 4
Results of the bacteriological examination of cloacal swabs with
regard to C. jejuni via the bacterial enrichment method

| | medica- | | number of cloacal swabs with | | |
|---|---|---|---|---|---|
| | tion | infection | C. jejuni/ | | |
| | with | with | total number of cloacal swabs | | |
| Group | PGS | C. jejuni | 14th day | 21st day | 28th day |
| 1 | yes | yes | 0/15 | 0/10 | 0/5 |
| 2 | yes | no | 0/15 | 0/10 | 0/5 |
| 3 | no | yes | 0/15 | 9/10 | 5/5 |
| 4 | no | no | 0/15 | 0/10 | 0/5 |

Bacteriological Examination of Cloacal Swabs

None of the cloacal swabs taken on the 14th day turned out to be Campylobacter-positive (Tab. 4). Bacteria were detected in none of the animals from groups 2 and 4 which had not been infected with C. jejuni.

However, a significant difference in the secretion rate could be detected between animals which had received PGS and had been infected with C. jejuni and animals which had not received PGS and had been infected with C. jejuni. These results show that, by administering PGS, a C. jejuni-infection can be avoided in chicken.

### Literature

EFSA (2005)

Scientific Report of the Scientific Panel on Biological Hazards on the request from the Commission related to Campylobacter in animals and foodstuffs., pp. 1-105 Annex to The EFSA Journal (2005).

EU (2003)

Directive 2003/99/EC of the European Parliament and of the Council of Nov. 17, 2003, for the monitoring of zoonoses and zoonotic agents, amending Council Decision 90/424/EEC and repealing Council Directive 92/117/EEC Glünder, G. (1993)

Campylobacter-Infektionen beim Geflügel—Epizootologie, Bedeutung and Bekämpfungsmoglichkeiten—. Archiv f. Geflügelkunde, 57, 241-248.

### EXAMPLE 6

The inventor of this invention contracted diarrhoea with vomiting through an infection, then took 2 heaped teaspoonfuls of PGS, stirred into yoghurt, and a decrease in symptoms was noted already after one hour.

1-18. (canceled)

19. A method of manufacturing a polyguanidine silicate comprising:
mixing a first aqueous solution comprising a polymeric guanidine salt with an inorganic or organic acid in a dissolved state with a second aqueous solution containing sodium and/or potassium silicate in a dissolved state,
whereby the polyguanidine silicate forms as a solid as well as a sodium and/or potassium salt of the inorganic or organic acid, which salt is present in dissolved form, whereupon the solid is separated.

20. The method of claim 19 wherein the polymeric guanidine salt is a polymeric bisguanidine salt.

21. The method of claim 19 wherein the polymeric guanidine salt is obtained by reacting a guanidine salt with an alkylene diamine and/or an oxyalkylene diamine.

22. The method of claim 19 wherein the polymeric guanidine salt is obtained via a reaction in which, per mole of diamine (sum of alkylene diamine and oxyalkylene diamine), 0.8 to 1.2 moles of guanidine salt are used.

23. The method of claim 21 wherein the polymeric guanidine salt is obtained via a reaction in which the alkylene diamine and the oxyalkylene diamine are used at a molar ratio of between 4:1 and 1:4.

24. The method of claim 21 wherein amino groups of the alkylene diamine and/or the oxyalkylene diamine are terminal.

25. The method of claim 21 wherein the alkylene diamine has the general formula NH₂(CH₂)ₙNH₂, wherein n is an integer between 2 and 10, in particular 6.

26. The method of claim 21 wherein the oxyalkylene diamine has the general formula NH₂[(CH₂)₂O)]ₙ(CH₂)₂NH₂, wherein n is an integer between 2 and 5, in particular 2.

27. The method of claim 21 wherein triethylene glycol diamine (relative molecular mass: 148), polyoxypropylene diamine (relative molecular mass: 230) and/or polyoxyethylene diamine (relative molecular mass: 600) is/are provided as the oxyalkylene diamine.

28. The method of claim 19 wherein an average molecular mass of the polymeric guanidine salt ranges between 500 and 3,000.

29. The method of claim 21 wherein a hydrochloride is provided as the salt of the guanidine.

30. The method of claim 19 wherein water glass is provided as the aqueous solution of a sodium and/or potassium silicate.

31. The method of claim 19 wherein the polyguanidine silicate is used as a biocidal agent.

32. The method of claim 19 wherein the polyguanidine silicate is used as an additive with biocidal activity, in particular in foodstuffs and animal feed.

33. The method of claim 19 wherein the polyguanidine silicate is used in fish breeding.

34. A polyguanidine silicate manufactured according to claim 1.

35. A drug composition comprising the polyguanidine silicate manufactured according to claim 1, wherein the polyguanidine silicate is a drug substance.

36. The drug composition of claim 35 for use in veterinary medicine.

37. The drug composition of claim 35 for use in fighting infections.
